(19) United States
(12) Patent Application Publication
(10) Pub. No.: US 2008/0314386 A1
(43) Pub. Date: Dec. 25, 2008

(54) Ventilation device for reducing hyperventilation

(75) Inventors: Helge Myklebust, Stavanger (NO); Kenneth George Morallee, Orpington (GB)

Correspondence Address: DORSEY & WHITNEY LLP;INTELLECTUAL PROPERTY DEPARTMENT, SUITE 3400, 1420 FIFTH AVENUE, SEATTLE, WA, 98101, US

(73) Assignee: Laerdal Medical AS, Stavanger (NO)

(21) Appl. No.: 11/821,588

(22) Filed: Jun. 21, 2007

(51) Int. Cl. A61M 16/00

(52) U.S. Cl. 128/205.15; 128/205.17

(57) ABSTRACT

A ventilation device is disclosed including a self-inflating bag which when compressed produces an outgoing flow, an inlet and an outlet and at least one valve connected to the inlet and/or outlet. The valve is disposed to control the flow out of the air outlet. One or more sensors positioned to sense air flow through the inlet or outlet produce outputs that are used by a controller to control opening and closing of the at least one valve to reduce hyperventilation.

### TECHNICAL FIELD

The invention relates to devices for providing emergency ventilation.

### BACKGROUND OF THE INVENTION

When a patient has little or no ability to breathe, such as during cardiac arrest, a rescuer will typically ventilate the patient by providing oxygen/air at regular intervals through the patient's mouth and airways. Ventilation of the patient is often combined with chest compressions to provide circulation of blood in the patient. Research has shown that the intervals and rates of ventilation are essential for the outcome of the resuscitation session.

Animal experiments have demonstrated the relationship between ventilation rates and brain tissue oxygenation. If ventilation rates are too high or low, tissue oxygenation is compromised, as shown by the data below from the Resuscitation Science Symposium. Circulation Vol 114; No 18, Oct. 31, 2006 (Abstracts from Resuscitation Science Symposium, November 2006).

The data of Table 1 from Idris et al (abstract 109) and Lurie et al (abstract 35) reflects brain tissue oxygen pressure measured using a Licox probe at different ventilation rates.

TABLE 1
Brain Tissue Oxygen Pressure v. Ventilation Rate

| | Brain Tissue O₂ | |
|---|---|---|
| Vent Rate [BPM] | [mmHg] | EtCO₂ [mmHg] |
| 2 | 0.5 +/− 0.1 | |
| 6 | 11 +/− 4 | 42 +/− 5 |
| 7 +/− 2 | 15 +/− 10 | 40 +/− 1 |
| 10 | 3.9 +/− 0.6 | |
| 12 | 3 +/− 3 | 31 +/− 4 |

The data suggest that there is an optimal ventilation rate, which causes a favorable brain tissue oxygenation in these models of low blood flow. With too low ventilation rates, apparently too little oxygen is circulated and the brain is harmed. With too high ventilation rates, too much carbon dioxide is removed from the blood which causes contraction of cerebral arteries, increased cerebral vascular resistance, and consequential reduced cerebral blood flow and cerebral tissue oxygenation. Irreversible cell damage is likely when tissue oxygenation drops below 10 mmHg. Under conditions of normal flow and fixed rate of 12 breaths per minute (bpm), brain tissue oxygenation was found to be in the range of 20-40 mmHg. In the low blood flow model above, 6-8 breaths per minute appears to be optimal with respect to cerebral tissue oxygenation.

In clinical practice, for patients in cardiac arrest or trauma, hyperventilation with rates higher than 6-8 bpm is standard. Reports show that ventilation rates in the range of 20-40 bpm are quite common during cardiac arrest. Such high ventilation rates have two known adverse effects. One effect is the excessive removal of carbon dioxide as mentioned above. The other effect is reduced cardiac preload, because ventilations result in increased airway and thoracic pressures. With elevated thoracic pressure, right side preload is compromised. With elevated airway pressure, right side preload is also compromised. Compromised preload means that less blood flows into the heart, hence less flow can be delivered out from the heart, and the effect is even lower perfusion pressures.

In order to reduce the incidence of hyperventilation, several mitigations have been tried, but with limited effect. These include visual and audible feedback and training. Other possibilities of preventing hyperventilation in low blood flow states include the use of automatic ventilators. These are limited in their application because of cost and complexity as well as size and logistic challenges. Feedback to the user (e.g. flashing lights or voice prompts) can help, but has so far have only demonstrated some improvement.

In view of the foregoing, it would be an advancement in the art to provide a ventilation device for effectively reducing hyperventilation during emergency ventilation.

### SUMMARY OF THE INVENTION

In one aspect of the invention there is provided a ventilation device comprising a self-inflating bag, which, when compressed, produces an outgoing flow. The bag includes an inlet allowing air to be drawn into the bag and an outlet permitting the outgoing flow. At least one valve is connected to the inlet and/or outlet to control air flow therethrough. The self-inflating bag may be any kind of bag which self inflates, for example, the type typically used in the art to ventilate patients. The valve connected to the inlet and/or the outlet may be a one-way valve, a two-way valve or a combination of such valves.

In another aspect of the invention, the valve is connected to the outlet and is a two-way valve. The two-way valve may also be embodied as a combination of two one-way valves of different flow directions. There may also be a second valve connected to the inlet, and in other embodiments there may be several valves connected to the outlet and/or inlet.

In another aspect of the invention, the valve or valves are disposed to control the time period between two succeeding bag compressions, i.e., the ventilation rate. The ventilation rate may be defined as the time between the maxima of a number of consecutive ventilations. There may also be set a minimum volume threshold for each ventilation. Accordingly the valve or valves may be controlled to meter the volume of air as well.

In one embodiment the ventilation device comprises a controller for controlling the valve(s). The ventilation device may include a sensor positioned to detect flow through the outlet 13. The sensor is connected to the controller and may be connected to an indicator. The sensor may be a CO₂ sensor, a ventilation sensor or a combination of these. The ventilation sensor may be disposed to measure the rate of ventilation, and/or the volume of each ventilation

The invention will now be described in more detail by means of examples of possible embodiments and with reference to the accompanying Figures.

### BRIEF DESCRIPTION OF THE DRAWINGS

FIG. 1 illustrates a ventilation device in accordance with an embodiment of the present invention.

FIG. 2 illustrates a ventilation device with an adjustable inlet restriction in accordance with an embodiment of the present invention.

FIG. 3 illustrates a ventilation device with a separate reservoir for expired air in accordance with an embodiment of the present invention.

FIG. 4 illustrates an alternative embodiment of the ventilation device of FIG. 2 in accordance with an embodiment of the present invention.

FIG. 5 illustrates another alternative embodiment of the ventilation device of FIG. 2 in accordance with an embodiment of the present invention.

### DETAILED DESCRIPTION OF PREFERRED EMBODIMENTS

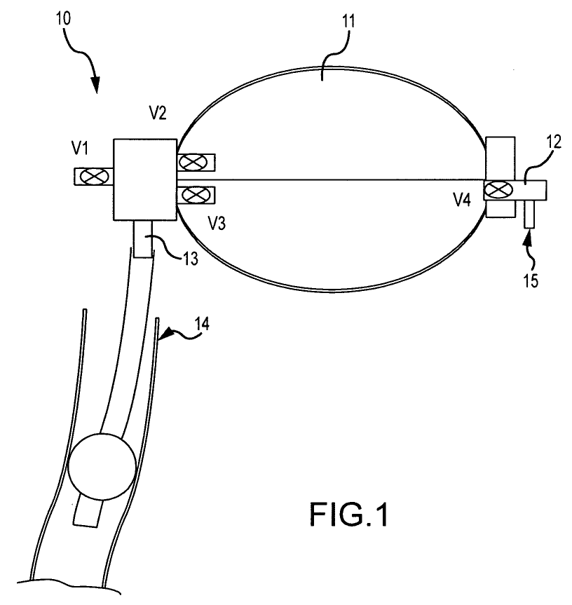

In FIG. 1, a ventilation device 10 includes a self-inflating bag 11, which, when compressed, produces an outgoing flow. The bag 11 includes an inlet 12 and an outlet 13, and at least one valve V1, V2, V3, V4 connected to the inlet 12 and/or outlet 13, the valves are disposed to control the flow out of the air outlet or into the air inlet.

The outlet 13 is connected to a secured airway 14, such as a mask, tube, combitube, laryngeal mask, or other suitable means which enables transport of air in and out of the airways without leakage. An oxygen source 15 may be connected to the inlet 12 to supply oxygen to the ventilation device and the patient's airways.

The Figure illustrates four valves, where V1, V2, V3 are connected to the outlet and V4 is connected to the inlet. In other embodiments, there may be other numbers and combinations of valves. The valves may, for example, be a valve V1 for controlling release of expired air to ambient, valve V3 for letting expired air into the bag 11, and valve V2 for letting air from the bag 11 into the outlet 13 and to the patient's airways. In some embodiments, the ventilation device 10 only comprises valves V1, V2 and V4, with valve V3 being omitted.

In some embodiments, the valve (or valves) is disposed to control the time period between two consecutive bag compressions to control the ventilation rate. The allowable ventilation rate may depend on the ventilation volume. For example a higher ventilation rate may be permissible if the ventilation volume is low compared to if the ventilation volume is high. Clinically, it is the product of ventilation rate and volume which has an impact on intrathoracic pressures and on gas exchange.

In one embodiment, the valve V4 connected to the inlet 12 and restricts air flow into the bag 11 through the inlet 12. This will increase the time used to fully inflate the bag, thus increasing the time period between two consecutive bag compressions. The restriction of valve V4 may be constant or adjustable. In the case of an adjustable restriction, the adjustment of the flow can be performed manually or automatically. Because different bag types have different elasticities and volumes, the restrictor may advantageously be calibrated according to a time constant for self inflation. Bags made of silicone have been found to have good stability over time and temperature with respect to elastic properties.

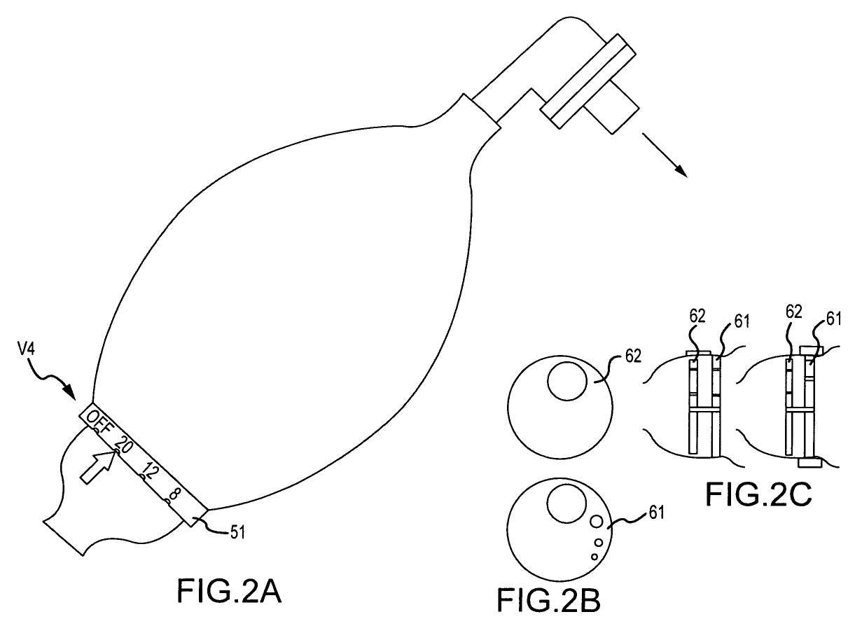

FIGS. 2A-2C illustrate an embodiment having a valve V4 providing an adjustable restriction. The user can operate a dial 51 disposed as part of the inlet valve assembly V4 in order to set the maximum possible number of self-inflations per minute. By operating the dial 51, the user rotates a disc in order to select between a number of restrictions or holes in a disc 61, through which the inlet air must pass. The smallest hole is used for the smallest rate of self-inflation, and the largest hole is used when self-inflation is not to be limited by restricting inlet airflow. In one embodiment, the dial will have four different settings: Off, 20, 12 and 8 self inflations per minute which correspond to 4 holes of decreasing diameter in a disk 61 which is connected to the dial 51. This disk 61 overlaps the disk 62, which has just one hole, which may have a diameter about equal to the largest diameter hole of the disc 61. FIG. 2B shows an example of two such disks 61, 62, which can be disposed in parallel and one of the discs 61, 62, rotated relative each other.

As an alternative to dial control of self-inflation, or other manually adjustable self inflation, the ventilation device 10 may comprise a controller for controlling the operation of one or several of the valves. Or the controller can be programmed to adjust the degree of restriction of valve V4 embodied as a restrictor for inlet air.

In another embodiment, the valve V4 which is connected to the inlet is an on/off valve that is switched on or off by the controller. This enables control of the number of self inflations per unit time. The timing control of the on/off valve can be set to limit the maximum set number of self inflations per minute.

The ventilation device 10 may in one embodiment comprise a sensor connected to the controller and/or to an indicator and positioned to sense air flow to and from the outlet 13. The sensor may be a CO₂ sensor, a ventilation sensor, a combination of these, or other sensors for providing information with respect to the ventilation of a patient.

In one embodiment, the ventilation sensor is positioned in an airway adapter positioned to sense flow through the outlet 13 or airway 14. In other embodiments, the ventilation sensor is integrated into a mask placed over a patient's face in fluid communication with the outlet 13.

In some embodiments, the sensor includes a restriction in the outlet 13 or airway 14, and a pressure sensor for measuring the pressure drop over this restriction. The pressure sensor(s) may in this case be placed in the ventilation device in or by an airway adapter, such as an adapter securing the airway 14 to the ventilation device. The flow rate can then be calculated as proportional to the square-root of the pressure drop. By integrating the flow the ventilation volume is found.

Alternative ventilation sensors may be constituted by means other than differential pressure monitoring, such as monitoring temperature fluctuations in the airways, which indicate whether the air is coming in or out of the person. Alternatively, a single pressure transducer, which measures the airway pressure inside the airway adapter may be used to enable detection of ventilation events and associated pressure profiles. In other embodiments, the motion of small turbines positioned in the airway may be used to sense ventilation. In still other embodiments, impedance measurements of the chest may be used to indicate the air volume in the lungs.

In one embodiment, the controller is programmed to control the opening/closing of the valve, such as one or more of the valve V1 to V4, when the time between two, or some other number, of operations of the ventilation device exceeds a pre-set rate threshold and opening the valve when the time between two, or some other number of operations, is lower than the pre-set rate threshold. The controller may also be programmed to control the opening/closing of the valve based on measurements of ventilation volume and rate.

The basis for controlling timing of compressions using the valve or valves may, for example, be provided by a sensor set to measure an actual rate of ventilation.

In one embodiment the valve V2 is an on/off valve connected to the outlet and controlling air flow from the outlet. Switching of the valve V2 is controlled by the controller. The timing of the on/off valve V2 may be controlled to achieve a maximum number of ventilations (compressions of the bag) per minute. The timing may be based on the measurement of the ventilation rate by a sensor coupled to a controller as described above. When the actual ventilation rate is less than the set maximum ventilation rate, the valve V2 is kept fully open by the controller. When the ventilation rate exceeds the set maximum value, the valve V2 will close, and open for ventilation after a delay that brings the ventilation rate within the desired range, i.e. below the set maximum value of the ventilation rate. The on/off valve V2 may be disposed as a fully mechanical solution, using energy from the operation of the bag or energy from the oxygen source 15 to operate and control the valve. The on/off valve may comprise a set function, where the user can set the maximum allowable rate of self inflation, for example between 6 and 16 ventilations per minute or some other value.

The on-off valve may also be battery operated, where energy in the battery is used to operate and control the valve, and where the on-off valve is further disposed with a selector to set the desired maximum number of self inflations per minute.

In concert with the battery operated on-off valve, the bag can be provided with an indicator. This can be a display, which indicates the actual rate of ventilation as a number, or colored lights, where the green light indicates that the actual ventilation rate is appropriate, a yellow light indicates that the actual rate is becoming too low or too high, and a red light indicates that the actual rate is too low or too high.

In some embodiments the controller is programmed to control one or more of the valves V1 to V4 based on the CO₂-level of the air expired by the patient. This can, for example, be used to increase or decrease ventilation rate to get the CO₂ level within a desired range. Some embodiments of the invention deliver gas from a source with a predetermined composition, or by taking advantage of the gas in the expired air of either the patient or the rescuer. Although maintaining proper ventilation rates and volumes is beneficial, it may be insufficient without also maintaining proper CO₂ levels. For example, ventilation rates of up to 12 bpm seem to be safe with respect to preload, but may still be too high to maintain normocapnia. Accordingly, the amount of CO₂ delivered to the patient may be increased in some embodiments of the invention.

Table 2 from International Volcanic Hazard Network (http://www.esc.cam.ac.uk/ivhhn/guidelines/gas/co2.html) indicates at which levels of concentration of CO₂ becomes dangerous.

TABLE 2
Health Effects of Carbon Dioxide

| Exposure | |
|---|---|
| limits | |
| (% in | |
| air) | Health Effects |
| 2-3 | Unnoticed at rest, but on exertion there may |
| | be marked shortness of breath |
| 3 | Breathing becomes noticeably deeper and |
| | more frequent at rest |
| 3-5 | Breathing rhythm accelerates. Repeated |
| | exposure provokes headaches |
| 5 | Breathing becomes extremely laboured, |
| | headaches, sweating and bounding pulse |
| 7.5 | Rapid breathing, increased heart rate, |
| | headaches, sweating, dizziness, shortness of |
| | breath, muscular weakness, loss of mental |
| | abilities, drowsiness, and ringing in the ears |
| 8-15 | Headache, vertigo, vomiting, loss of |
| | consciousness and possibly death if the |
| | patient is not immediately given oxygen |
| 10 | Respiratory distress develops rapidly with |
| | loss of consciousness in 10-15 minutes |
| 15 | Lethal concentration, exposure to levels |
| | above this are intolerable |
| 25+ | Convulsions occur and rapid loss of |
| | consciousness ensues after a few breaths. |
| | Death will occur if level is maintained. |

The composition of air before and after breathing is shown in Table 3 below. Gas composition, from http://www.pdh-odp.co.uk/GasLaws.htm.

TABLE 3
Air Composition

| | DRY AIR | | HUMIDIFIED AIR | | ALVEOLAR AIR | | EXPIRED AIR | |
|---|---|---|---|---|---|---|---|---|
| GASES | mmHg | % | mmHg | % | mmHg | % | mmHg | % |
| Nitrogen | 600.2 | 78.98 | 563.4 | 74.09 | 569.0 | 74.9 | 566.0 | 74.5 |
| Oxygen | 159.5 | 20.98 | 149.3 | 19.67 | 104.0 | 13.6 | 120.0 | 15.7 |
| Carbon dioxide | 0.3 | 0.04 | 0.3 | 0.04 | 40.0 | 5.3 | 27.0 | 3.6 |
| Water vapor | 0.0 | 0.0 | 47.0 | 6.20 | 47.0 | 6.2 | 47.0 | 6.2 |

Aufderheide (Circulation, Apr. 27, 2004) demonstrated that a CO₂ level of 5% in the inspired air (rate 30 per minute) resulted in normocapnia both looking at blood gases and at ETCO₂. In this experiment, 5% CO₂ came from a dedicated gas source.

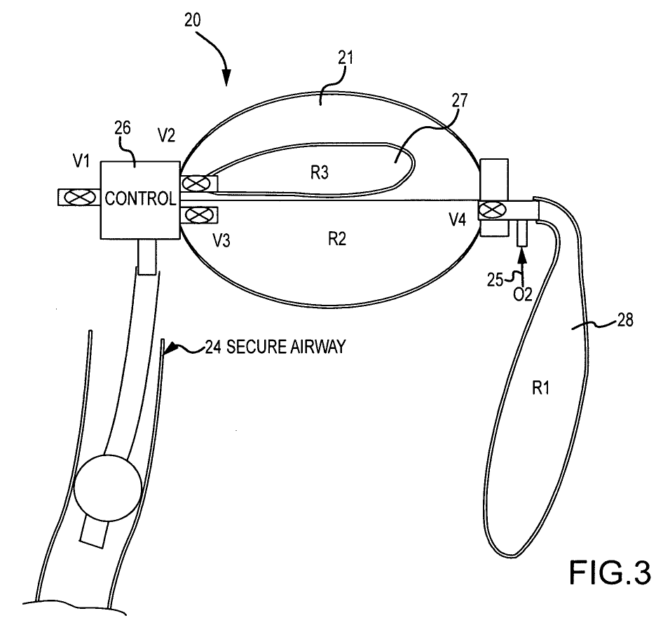

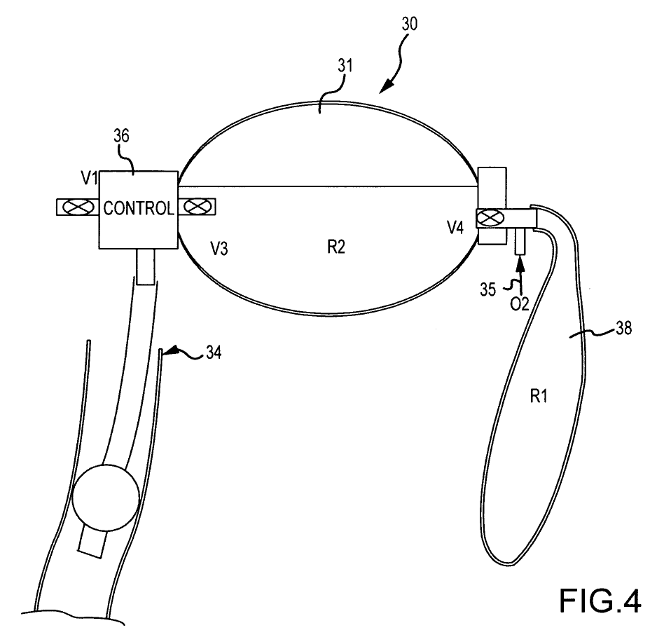

FIGS. 3 and 4 illustrate two examples of a ventilation device 20, 30 according to an embodiment of the invention with a reservoir for expired air. In FIG. 3, there is a separate reservoir 27 for the expired air which is let into the reservoir through valve V2 connected to the outlet. Valve V2 may be a two-way valve or a combination of two one-way valves. A valve V1 may be disposed to let expired air to the ambient, and a valve V3 may be disposed to let fresh air from bag 11, 21, 31 through the outlet. In FIG. 4, there is no separate reservoir for the expired air, but expired air may be let back into the bag 31 through valve V3 which may be a two-way valve. Alternatively valve V3 may be embodied as two one-way valves as in the embodiment in FIG. 3. Valves V2, V3 may be mechanically operated or electrically controlled. The dedicated reservoir may be disposable for single patient use. The control mechanism may be battery operated, where the sensor is connected to a microprocessor control unit programmed to control the valves and hence the direction of air flow.

An oxygen source 25, 35 may be provided and disposed such that a continuous flow of oxygen is collected in an O₂ reservoir 28, 38.

The ventilation device 20, 30 may comprise a controller 26, 36 as described in connection with FIG. 1. The controller may be programmed to operate the valve V2 such that none, some, or all of the expired air is directed back to a reservoir placed inside the bag volume 21, 31. For instance, the controller can be set to allow a proportion of a number of ventilations to be supplemented with recycled expired air. In such an embodiment, the expired air within the reservoir bag will be delivered back to the patient again through valve V2 when the bag is operated.

In some embodiments, the bag 11, 21, 31 is divided into two separate compartments or volumes. As shown in FIG. 3, the upper volume is in fluid communication with valve V2 whereas the lower volume is in fluid communication with valves V3 and V4, which allows fresh air into the lower volume. Accordingly, when the bag is compressed, expired air can be expelled from the upper volume through valve V2 whereas fresh air is expelled through valve V3. The valves V2 and V3 may be opened or closed by the controller to control the proportion of fresh and expired air. The controller may be connected to a sensor which measures ventilation rate, and the controller may operate the valves V2 and V3 in such a way that the proportion of recycled air has a particular relationship with, for example, proportional to, the applied ventilation rate.

In an alternative embodiment, the controller may be connected to a carbon dioxide sensor positioned in fluid communication with the airway 24 to sense the carbon dioxide of expired air. In such embodiments, the controller may control valves V2 and V3 such that the proportion of recycled air is chosen to achieve a desired set level of carbon dioxide in the exhaled air.

In FIGS. 1, 3, and 4, the controller 26, 36 is disposed on the patient side of the self-inflating bag, and connects to each of the different valves V1 to V4. In normal operation, with actual carbon dioxide or ventilation rate within the desired range, valve V3 may open when the bag is compressed and close when the bag is released for self inflation where at the same time V1 opens to allow expired air to the ambient. When the control unit determines recycling of expired air is needed, the valve V2 will be opened for a time period such that some or all of the expired air is collected before V2 closes and V1 opens to permit the remaining expired air to flow to ambient. To recycle expired air, the control unit now opens V2 upon compression of the bag, and may open V3 at some point to allow delivery of mixed recycled and oxygenated air. During self inflation, the valve V4 which is connected to the inlet will be opened to allow oxygenated air to enter the bag.

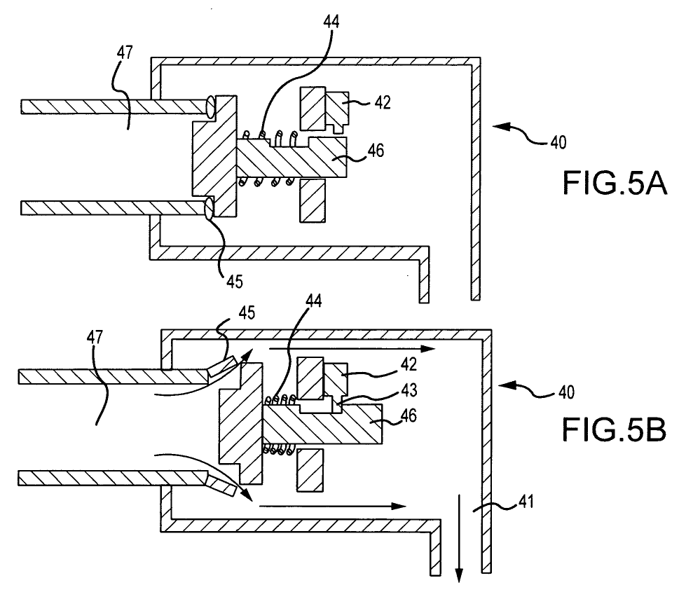

FIG. 5 illustrates a hyperventilation prevention mechanism 40 for use in a ventilation device according to an embodiment of the invention, such as is described hereinabove, however it may be used with conventional ventilators as well. The hyperventilation prevention mechanism 40 is disposed within a housing 41 having an inlet 47 and an outlet. The inlet 47 is adapted to connect to the outlet of a ventilation device having a self-inflating bag, such as shown in FIGS. 1 through 4. The hyperventilation prevention mechanism 40 includes a threshold mechanism with a piston which is arranged to require an elevated threshold pressure before allowing ventilation under conditions of hyperventilation. The threshold mechanism may be mechanically powered and controlled, may be electrically powered and controlled, or may be subject to any combination of electrical and mechanical power and control.

Sensing of hyperventilation may be performed by a pressure sensor, for example an electronic pressure sensor connected to the patient side of the self-inflating bag. A microcontroller may be provided with a connection to the pressure sensor and comprise algorithms to calculate an approximate ventilation rate. When the approximate ventilation rate exceeds a predefined value, the microcontroller may output a signal to a bi-stable solenoid 42, which releases a locking member 43 which releases the piston 46. The piston 46 is driven by a spring 44, which may be non-linear. In normal conditions, that is without hyperventilation, the piston 46 may be locked in open position by the locking member 43 of the solenoid 42. This situation is illustrated in FIG. 5B. As hyperventilation is detected, the solenoid 42 releases, and the non-linear spring 44 brings the piston 46 into a locked position. This situation is illustrated in FIG. 5B. With the next attempted ventilation, the piston 46 will stay in the locked position until the force generated by the air pressure generated by squeezing the bag moves the piston 46 to an open position. A flexible seal 45 may be disposed between the outlet and the piston 46, such that the piston 46 must travel a distance before the seal opens. When the microcontroller senses that hyperventilation has ceased, the output of the controller may trigger the solenoid 42 such that the locking member 43 engages the piston 46 such that the piston 46 remains in the open position.

What is claimed is:

1. A ventilation device comprising:
a self-inflating bag having an inlet and an outlet, the outlet adapted to be placed in fluid communication with a person's airway, the self-inflating bag coupled to the outlet to produce an outgoing flow from the outlet upon compression;
a valve disposed to control flow of air through at least one of the outlet and the inlet;
a sensor sensing air flow through at least one of the inlet and the outlet;
a controller coupled to the valve and operable to control opening of the valve according to an output from the sensor.

2. The ventilation device of claim 1, wherein the sensor is an air flow sensor.

3. The ventilation device of claim 1, wherein the sensor is a carbon dioxide sensor.

4. The ventilation device of claim 3, wherein the controller is further operable to control air flow into the bag through the outlet during self inflation of the bag according to the output from the carbon dioxide sensor.

5. The ventilation device of claim 1, wherein the valve is a first valve and further comprising a second valve, the first valve in fluid communication with an inner reservoir positioned within the self-inflating bag and the second valve in fluid communication with the outlet and a volume defined by the self-inflating bag.

6. The ventilation device of claim 1, wherein the valve is a first valve and further comprising a second valve, the first valve disposed to control flow through the outlet and the second valve disposed to control flow through the outlet.

7. The ventilation device of claim 6, wherein the sensor is an air flow sensor and further comprising a carbon dioxide sensor coupled to the controller, the controller operable to control opening of the first valve according to the carbon dioxide sensor and to control opening of the second valve according to the air flow sensor.

8. The ventilation device of claim 1, wherein the valve is a two-way valve.

9. The ventilation device of claim 1, wherein the controller is operable to control opening of the valve to control a time period between consecutive compressions.

11. The ventilation device of claim 9, wherein the controller is programmed to close the valve when the time between consecutive compressions of the self-inflating bag exceeds a pre-set rate threshold and opening the valve when the time between consecutive compressions of the self-inflating bag are lower than the pre-set rate threshold.

12. The ventilation device of claim 1, wherein the sensor is an air flow sensor and wherein the controller is operable to control the opening and closing of the valve according to a ventilation volume and rate determined from the output of the air flow sensor.

13. The ventilation device of claim 1, wherein the valve is an on/off valve connected to the outlet and wherein the controller is operable to switch the valve on and off.

14. The ventilation device of claim 1, further comprising an oxygen source in fluid communication with the inlet.

15. The ventilation device of claim 1, further comprising an indicator coupled to the sensor, the indicator visually indicating at least one of flow rate and compression rate relative to a threshold.

16. The ventilation device of claim 1, further comprising:
a pressure sensitive valve positioned in fluid communication with the outlet and disposed to control flow out of the outlet that exceeds a threshold pressure; and
a lock selectively locking the pressure sensitive valve open, the controller coupled to the lock and operable to cause the lock to lock the pressure sensitive valve open upon detecting an output from the sensor indicating that hyperventilation is not occurring.

17. A method for ventilating a patient comprising:
placing an outlet in fluid communication with a patient's airway;
compressing a self-inflating bag in fluid communication with the outlet to force air into the patient's airway;
measuring airflow through the outlet; and
controlling opening of a valve controlling air flow at least one of into and out of the self-inflating bag according to the measured airflow.

18. The method of claim 17, further comprising selectively permitting air from the patient to enter the self-inflating bag through the outlet according to the measured air flow.

19. The method of claim 17, further comprising selectively permitting air from the patient to enter the self-inflating bag through the outlet according to an output from a carbon dioxide sensor in fluid communication with air flow from the patient's airway.

20. The method of claim 17, further comprising:
positioning a pressure sensitive valve in fluid communication with the outlet and disposed to control flow therethrough;
locking the pressure sensitive valve in an open position when the measured airflow indicates that hyperventilation is not occurring;
ceasing to lock the pressure sensitive valve in the open position when the measured airflow indicates that hyperventilation is occurring.
